(19) United States
(12) Patent Application Publication
(10) Pub. No.: US 2026/0072006 A1
(43) Pub. Date: Mar. 12, 2026

(54) ELECTRODE ARRAY DEVICE

(71) Applicant: SAMSUNG ELECTRONICS CO., LTD., Suwon-si (KR)

(72) Inventors: JOONSEONG KANG, Suwon-si (KR); KITAE PARK, Suwon-si (KR); NAGJONG KIM, Suwon-si (KR); CHANG-WOO SHIN, Suwon-si (KR); SUNGHAENG LEE, Suwon-si (KR); HYEOKKI HONG, Suwon-si (KR)

(73) Assignee: SAMSUNG ELECTRONICS CO., LTD., Suwon-si (KR)

(21) Appl. No.: 19/220,962

(22) Filed: May 28, 2025

(30) Foreign Application Priority Data
Sep. 6, 2024 (KR) 10-2024-0121833

(51) Int. Cl. G01N 33/483

(52) U.S. Cl. CPC G01N 33/4836

(57) ABSTRACT

An electrode array device includes a first electrode array including a plurality of electrodes configured to contact a target, a first active chip configured to perform, with the first electrode array, a first measurement function on the target using a first frequency range and receive a first input signal corresponding to the first frequency range from the first electrode array, a second active chip, which is structurally different from the first active chip, configured to perform, with the first electrode array, a second measurement function on the target using a second frequency range that is different from the first frequency range and receive a second input signal corresponding to the second frequency range from the first electrode array, and a signal path selector.

### CROSS-REFERENCE TO RELATED APPLICATION

This application is based on and claims priority to Korean Patent Application No. 10-2024-0121833, filed on Sep. 6, 2024, in the Korean Intellectual Property Office, the disclosure of which is incorporated by reference herein in its entirety.

### BACKGROUND

### 1. Field

The disclosure relates to an electrode array device.

### 2. Description of Related Art

A multi-electrode array (MEA) may include a large number of ultra-small, high-density electrodes (e.g., thousands of electrodes), and an MEA system may measure an electrical signal input through electrodes or generate an output to the electrodes to cause a change in a target and/or around the target that the electrodes contact. Changes may be used to facilitate measurements. For example, an MEA system may be used to measure the electrical activities of living tissue (e.g., neural tissue) or for various applications (e.g., deoxyribonucleic acid (DNA) synthesis, DNA sequencing, a micro-chemical sensor, a micro-odor sensor, etc.).

### SUMMARY

Additional aspects will be set forth in part in the description which follows and, in part, will be apparent from the description, or may be learned by practice of the presented embodiments of the disclosure.

According to an aspect of the disclosure, an electrode array device may include a first electrode array including a plurality of electrodes configured to contact a target, a first active chip configured to perform, with the first electrode array, a first measurement function on the target using a first frequency range and receive a first input signal corresponding to the first frequency range from the first electrode array, a second active chip, which is structurally different from the first active chip, configured to perform, with the first electrode array, a second measurement function on the target using a second frequency range that is different from the first frequency range and receive a second input signal corresponding to the second frequency range from the first electrode array, and a signal path selector configured to selectively connect at least one electrode of the plurality of electrodes to the first active chip in the case that the first active chip is to perform the first measurement function or to the second active chip in the case that the second active chip is to perform the second measurement function.

The signal path selector may be further configured to connect a first electrode of the plurality of electrodes to the first active chip in the case that the first measurement function is to be performed, and connect a second electrode of the plurality of electrodes that is different from the first electrode to the second active chip in the case that the second measurement function is to be performed.

The signal path selector may be further configured to connect a first electrode of the plurality of electrodes to the first active chip in the case that the first measurement function is to be performed, and connect the first electrode of the plurality of electrodes to the second active chip in the case that the second measurement function is to be performed.

The signal path selector may include a path switch configured to selectively connect the at least one electrode to the first active chip in the case that the first measurement function is to be performed and the second active chip in the case that the second measurement function is to be performed.

The signal path selector may include an input amplifier configured to amplify an input signal input from the at least one electrode, and an output amplifier configured to amplify an output signal output from the first active chip or the second active chip.

The signal path selector may include a base signal path configured to fully connect, to the path switch, first signal terminals of the first active chip and second signal terminals of the second active chip, and the base signal path may include a plurality of partial signal paths.

The signal path selector may be further configured to connect a first partial signal path of the plurality of partial signal paths to the first electrode array, and connect a second partial signal path of the plurality of partial signal paths to a second electrode array that is different from the first electrode array, and the first partial signal path may be different from the second partial signal path.

The first frequency range may be in a range of 100 Hz to 20 kHz, and the second frequency range may be in a range of direct current (DC) to 100 Hz.

The first input signal may include an action potential (AP) signal, and the second input signal may include at least one of a local-field potential (LFP) signal, a potential of hydrogen (pH) and/or electrical conversion signal, an ion and/or electrical conversion signal, and a combination thereof.

The first active chip and the second active chip may be produced from separate semiconductor dies.

The first active chip, the second active chip, and the signal path selector may be configured as an active chipset, and the electrode array device may include an interposer connecting the active chipset to the first electrode array.

The interposer may include the plurality of electrodes.

The first active chip may be vertically stacked on the second active chip, and the signal path selector may be connected to the first active chip and the second active chip through a through silicon via (TSV).

The first electrode array may be on the first active chip.

According to an aspect of the disclosure, an electrode array device may include an electrode array including a plurality of electrodes configured to contact a target, a first active chip configured to perform, with the electrode array, a first measurement function on the target using a first frequency range, a second active chip configured to perform, with the electrode array, a second measurement function on the target using a second frequency range that is different from the first frequency range, a third active chip configured to perform, with the electrode array, a stimulation function on the target, and a signal path selector configured to selectively connect at least one electrode of the plurality of electrodes to the first active chip in the case that the first active chip is to perform the first measurement function, the second active chip in the case that the second active chip is to perform the second measurement function, and the third active chip in the case that the third active chip is to perform the stimulation function.

The signal path selector may be further configured to connect a first electrode of the plurality of electrodes to the first active chip in the case that the first measurement function is to be performed, and connect a second electrode of the plurality of electrodes that is different from the first electrode to the second active chip in the case that the second measurement function is to be performed.

The signal path selector may be further configured to connect a first electrode of the plurality of electrodes to the first active chip in the case that the first measurement function is to be performed, and connect the first electrode to the second active chip in the case that the second measurement function is to be performed.

The signal path selector may include a path switch configured to selectively connect the at least one electrode to the first active chip in the case that the first measurement function is to be performed, the second active chip in the case that the second measurement function is to be performed, and the third active chip in the case that the stimulation function is to be performed.

The signal path selector may include a base signal path configured to fully connect, to the path switch, first signal terminals of the first active chip, second signal terminals of the second active chip, and third signal terminals of the third active chip, and the base signal path may include a plurality of partial signal paths.

According to an aspect of the disclosure, an electrode array device may include an electrode array including a plurality of electrodes configured to contact a target, a first active chip configured to perform, with the electrode array, a first function on the target, a second active chip that is structurally different from the first active chip and configured to perform, with the electrode array, a second function on the target, the second function being different from the first function, a third active chip that is structurally different from the first active chip and the second active chip and configured to perform, with the electrode array, a third function on the target, the third function being different from the first function and the second function, and a signal path selector configured to selectively connect at least one electrode of the plurality of electrodes to the first active chip in the case that the first function is to be performed, the second active chip in the case that the second function is to be performed, and the third active chip in the case that the third function is to be performed

### BRIEF DESCRIPTION OF DRAWINGS

The above and other aspects, features, and advantages of certain embodiments of the disclosure will be more apparent from the following description taken in conjunction with the accompanying drawings, in which:

FIG. 1 is a diagram illustrating an example of a configuration of an electrode array device, according to one or more embodiments;

FIG. 2 is a diagram illustrating an example of the versatility of an active chipset, according to one or more embodiments;

FIGS. 3 and 4 are diagrams illustrating examples of a configuration of a signal path selector, according to one or more embodiments;

FIGS. 5A to 5C are diagrams illustrating examples of an application in which an electrode array is used for different functions, based on a time interval, according to one or more embodiments;

FIGS. 6A and 6B are diagrams illustrating examples of an application in which electrodes of an electrode array are used for different functions, based on a position in the electrode array, according to one or more embodiments;

FIGS. 7A to 7C are diagrams illustrating examples of an application in which electrodes of an electrode array are used for different functions, based on a time interval, according to one or more embodiments;

FIG. 8 is a diagram illustrating an example of packaging an electrode array device using an interposer, according to one or more embodiments;

FIG. 9 is a diagram illustrating an example of packaging an electrode array device using vertical stacking, according to one or more embodiments;

FIG. 10 is a diagram illustrating an example of the arrangement of signal path selectors of an electrode array device, according to one or more embodiments; and

FIG. 11 is a diagram illustrating an example of three or more active chips being used in an electrode array device, according to one or more embodiments.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

Although terms, such as first, second, and the like are used to describe various components, the components are not limited to the terms. These terms should be used only to distinguish one component from another component. For example, a first component may be referred to as a second component, or similarly, the second component may be referred to as the first component.

In the following description, when a component is referred to as being “above” or “on” another component, it may be directly on an upper, lower, left, or right side of the other component while making contact with the other component or may be above an upper, lower, left, or right side of the other component without making contact with the other component.

As used herein, the term “and/or” includes any and all combinations of one or more of the associated listed items. Expressions such as “at least one of,” when preceding a list of elements, modify the entire list of elements and do not modify the individual elements of the list.

It should be noted that if it is described that one component is “connected”, “coupled”, or “joined” to another component, a third component may be “connected”, “coupled”, and “joined” between the first and second components, although the first component may be directly connected, coupled, or joined to the second component.

The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

For example, the expression, “at least one of a, b, and c,” should be understood as including only a, only b, only c, both a and b, both a and c, both b and c, or all of a, b, and c.

In addition, terms such as “unit” and “module” described in the specification may indicate a unit that processes at least one function or operation, and this may be implemented as hardware or software, or may be implemented as a combination of hardware and software.

Operations of a method may be performed in an appropriate order unless explicitly described in terms of order. In addition, the use of all illustrative terms (e.g., etc.) is merely for describing technical ideas in detail, and the scope is not limited by these examples or illustrative terms unless limited by the claims.

Unless otherwise defined, all terms, including technical and scientific terms, used herein have the same meaning as commonly understood by one of ordinary skill in the art to which this disclosure pertains. Terms, such as those defined in commonly used dictionaries, should be construed to have meanings matching with contextual meanings in the relevant art, and are not to be construed to have an ideal or excessively formal meaning unless otherwise defined herein.

Hereinafter, example embodiments will be described in detail with reference to the accompanying drawings. The embodiments described below are merely exemplary, and various modifications are possible from these embodiments. In the following drawings, the same reference numerals refer to the same components, and the size of each component in the drawings may be exaggerated for clarity and convenience of description.

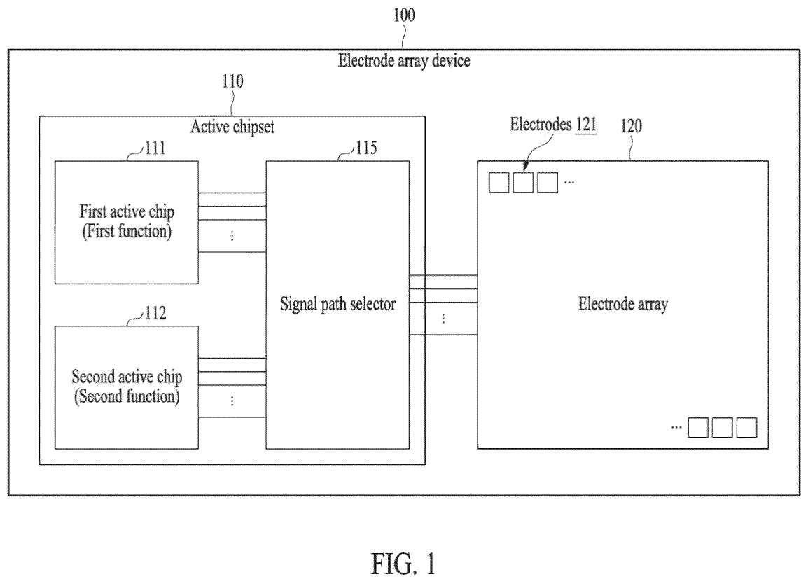

FIG. 1 is a diagram illustrating an example of a configuration of an electrode array device, according to one or more embodiments. Referring to FIG. 1, an electrode array device 100 may include an active chipset 110 and an electrode array 120.

The electrode array 120 may include electrodes 121 contacting a target. For example, the target may be, but is not limited thereto, biological tissue (e.g., neural tissue), deoxyribonucleic acid (DNA), a chemical substance, etc. The electrodes 121 may be arranged in an array shape but are not limited thereto. For example, the electrode array 120 may correspond to, but is not limited thereto, a multi-electrode array (MEA). An MEA system may perform a measurement function and/or a stimulation function on the target using a multi-electrode and a complementary metal-oxide semiconductor (CMOS) array configured as an ultra-small, high-density.

The active chipset 110 may perform a function in relation to the target using the electrode array 120. The function may correspond to various function categories and may be implemented utilizing various specifications. For example, the category of the function may include a measurement function and a stimulation function. An electrical signal of the target may be measured through the measurement function. The measurement function may correspond to a recording function. The target and/or an area around the target may be stimulated through the stimulation function. For example, the stimulation may include, but is not limited to, electrical stimulation, chemical stimulation, thermal stimulation, etc. The stimulation may be used to create a desired measurement environment. For example, the specification of the function may include, but is not limited to, a usage frequency range, a stimulation signal intensity, a usage power range, a number of used electrodes, etc. For example, the function may include a first measurement function using a first frequency range (e.g., a relatively high-frequency range, such as 100 hertz (Hz) to 20 kilohertz (kHz)) and a second measurement function using a second frequency range (e.g., a relatively low-frequency range, such as direct current (DC) to 100 Hz) that is different from the first frequency range.

The electrode array device 100 may use the active chipset 110 with general-purpose properties and the electrode array 120 with dedicated properties. The active chipset 110 may be designed to be available for other electrode arrays that are distinguished from the electrode array 120.

Due to the characteristics of the target and/or applications, there may be application-specific requirements for the electrode array 120 to perform a function in relation to the target. Due to the characteristic of the application-specific requirements for the electrode array 120, the electrode array 120 with dedicated properties may be used for each application.

For example, for recording and stimulating biological tissue, the electrode array 120 may be required to be capable of measuring electric potential and be a planar or three-dimensional (3D)-shaped array. For DNA synthesis, the electrode array 120 may be required to be capable of converting current or voltage into potential of hydrogen (pH) and be a planar-well shaped array. For DNA sequencing, the electrode array 120 may be required to be capable of converting pH into voltage and be a 3D-well shaped array. In the case of an ion sensor (e.g., an ion-sensor field-effect transistor (FET) (ISFET)), the electrode array 120 may be required to be capable of converting ion concentration into resistance. In the case of an olfactory sensor (e.g., an electrical nose (eNose)), the electrode array 120 may be required to be capable of converting the concentration of gas components into a surface acoustic wave (SAW). In the example of FIG. 1, the electrode array 120 that satisfies the above requirements may be used.

Due to the characteristics of the target and/or applications, there may be application-specific requirements for the active chipset 110 to perform a function in relation to the target. The active chipset 110 may be designed to universally satisfy the requirements described below.

For example, for recording and stimulating biological tissue, the active chipset 110 may be required to perform a recording function of local-field potential (LFP) and action potential (AP), and to perform a current and voltage stimulation function. For recording LFP and AP, the recording function for a certain frequency range (e.g., 100 Hz to 20 kHz) may be required. The stimulation function for a certain current (e.g., 2 nA) and a certain voltage (e.g., several mV) may be required. For example, for DNA synthesis, the active chipset 110 may be required to perform a current stimulation function and have massive multi-channel processing capability. For example, the stimulation function for a certain current (e.g., +tens of nA, tens of seconds) may be required. For DNA sequencing, the active chipset 110 may be required to perform a voltage-sensing function and have massive multi-channel processing capability. For example, DC voltage sensing may be required. For an olfactory sensor, the active chipset 110 may be required to perform a function of converting a frequency into time.

The active chipset 110 may include a first active chip 111, a second active chip 112, and a signal path selector 115. The first active chip 111 may perform a first function on the target using the electrode array 120. The second active chip 112 may be distinguished from the first active chip 111 and may perform a second function that is different from the first function on the target using the electrode array 120. Although FIG. 1 illustrates an example in which the active chipset 110 includes two active chips (e.g., the first active chip 111 and the second active chip 112), embodiments are not limited thereto. For example, the active chipset 110 may include three or more active chips.

The first active chip 111 and the second active chip 112 may include active elements to perform the first function and the second function, respectively. For example, when the first function is a measurement function, the first active chip 111 may include active measurement elements to perform the measurement function. When the second function is a stimulation function, the second active chip 112 may include active stimulation elements to perform the stimulation function.

The active chipset 110 may have general-purpose properties corresponding to a plurality of active chips, such as the first active chip 111 and the second active chip 112. According to one or more embodiments, the first function of the first active chip 111 and the second function of the second active chip 112 may correspond to the measurement function and the stimulation function, respectively. In this case, the active chipset 110 may be implemented with the electrode array 120 that is capable of performing measurement and stimulation functions and used for various applications that perform measurement and stimulation. According to one or more embodiments, the first function may correspond to the first measurement function using the first frequency range, and the second function may correspond to the second measurement function using the second frequency range that is distinguished from the first frequency range. In this case, the active chipset 110 may be implemented with the electrode array 120 capable of performing measurement and used for various applications that perform measurement using different frequency ranges (e.g., the first frequency range and the second frequency range).

For example, the first active chip 111 may perform the first measurement function of the first frequency range on the target and receive a first input signal corresponding to the first frequency range from the electrode array 120, and the second active chip 112 may perform the second measurement function of the second frequency range on the target and receive a second input signal corresponding to the second frequency range from the electrode array 120. For example, the first frequency range may be a relatively high-frequency range, such as 100 Hz to 20 kHz, and the second frequency range may be a relatively low-frequency range, such as DC to 100 Hz.

For example, among various signals generated from a nerve cell, an AP signal may have a relatively high-frequency range characteristic of a band of 100 kHz to 20 kHz, and an LFP signal may have a relatively low-frequency range characteristic of a band of DC to 100 Hz. In this case, the active chipset 110 may be universally used for a first application (e.g., a first measurement function) for measuring AP and a second application (e.g., a second measurement function) for measuring LFP. In this case, the first input signal may be the AP signal, and the second input signal may be the LFP signal. In addition, a pH/electrical conversion signal used for DNA sequencing and an ion/electrical conversion signal used for detecting ion components may have a low-frequency range characteristic of a band of DC to 100 Hz. For example, DNA sequencing may be performed in real time after DNA synthesis. In this case, the active chipset 110 may be universally used for the first application (e.g., a recording function) for recording AP and the second application (e.g., a measurement function) for measuring the pH/electrical conversion signal for DNA sequencing or the ion/electrical conversion signal for detecting ion components. In this case, the first input signal may be the AP signal, and the second input signal may be the pH/electrical conversion signal or an ion/electrical conversion signal. The pH/electrical conversion signal may refer to a signal in which pH is electrically converted, and the ion/electrical conversion signal may refer to a signal in which ion concentration is electrically converted.

When the first application and the second application further require the stimulation function, another active chipset further including a third active chip that performs the stimulation function as a third function may be used in addition to the first active chip 111 and the second active chip 112. For example, the third active chip may be used when the electrical stimulation is applied to a cell to cause electroporation for measuring intra-cellular potential in measuring a neural cell signal or when the electrical stimulation is applied around an electrode to cause a pH change during a real-time sequencing process after DNA synthesis.

Due to the general-purpose properties of the active chipset 110, the design of the electrode array device 100 may be facilitated, and the range of use of the active chipset 110 may be expanded. For example, the design of the electrode array device 100 may focus on the design of the electrode array 120. Accordingly, the development cost of the active chipset 110 may be reduced, and the technology development speed may be accelerated. The production cost of the active chipset 110 may be reduced due to mass production of the active chipset 110. By focusing on the development of the active chipset 110 during the initial development of the active chipset 110, the stability of the active chipset 110 may increase, and the maintenance of the active chipset 110 may be improved.

The first active chip 111 and the second active chip 112 may be produced from separate semiconductor dies. That is, the first active chip 111 may be produced from a first semiconductor die, and the second active chip 112 may be produced from a second semiconductor die. In other words, the first active chip 111 may be a separate chip from the second active chip 112. For example, different designs and/or processes may be applied to different semiconductor dies. Specifically, the first active chip 111 may be structurally different from the second active chip 112. In one or more embodiments, a third active chip may be provided. The third active chip may be produced from a semiconductor die that is different from the semiconductor dies used to produce the first active chip 111 and the second active chip 112. Thus, the third active chip may be structurally different from the first active chip 111 and the second active chip 112. More than three active chips may be provided, all of which may be produced from different semiconductor dies.

Higher performance may be achieved compared to a case in which different functions (e.g., the first function and the second function) are provided by a single active chip produced from one semiconductor die. For example, when the first active chip 111 performing the first measurement function corresponding to the first frequency range and the second active chip 112 performing the second measurement function corresponding to the second frequency range are used, higher performance may be achieved with a smaller area compared to a case in which a single active chip performing the measurement function of a single frequency range including the first frequency range and the second frequency range is used. The further the frequency ranges of interest (e.g., the first frequency range and the second frequency range) are, the greater the performance difference between a multi-active chip (e.g., the first active chip 111 and the second active chip 112) and a single active chip may be. When the first active chip 111 performing the measurement function and the second active chip 112 performing the stimulation function are used, since the first active chip 111 and the second active chip 112 are implemented separately, noise due to measurement and stimulation may be reduced, and measurement and stimulation performance may be improved.

More specifically, the first active chip 111 may be produced through a semiconductor die developed for the purpose of signal recording in a relatively high frequency range (e.g., 100 kHz to 20 kHz). The second active chip 112 may be produced through a semiconductor die developed for the purpose of signal recording in a relatively low frequency range (e.g., DC to 100 Hz). In this case, an individual semiconductor die developed for each frequency range (e.g., 100 kHz to 20 kHz and DC to 100 kHz) may exhibit higher recording performance in each frequency range, compared to a single semiconductor die developed for the purpose of signal recording in the entire frequency range (e.g., DC to 20 kHz). In addition, the total implementation area of the semiconductor dies for each individual frequency range may be narrower than that of a single semiconductor die for the entire frequency range. Additionally, developing the individual semiconductor die for each frequency range may be easier than developing a single semiconductor die for the entire frequency range.

In addition, the first active chip 111 may be produced through a semiconductor die developed for the purpose of recording, and the second active chip 112 may be produced through a semiconductor die developed for the purpose of stimulation. For example, the first active chip 111 may be produced through a semiconductor die manufactured using a predetermined semiconductor manufacturing process (e.g., a low-voltage (LV) process) to operate with a supply power source of relatively low voltage (e.g., 0.5 V to 1.0 V) for recording and have low power and low noise characteristics. The second active chip 112 may be produced through a semiconductor die manufactured using a predetermined semiconductor manufacturing process (e.g., a high-voltage (HV) process) to operate with a supply power source of relatively high voltage (e.g., 3.3 V to 12 V) required for stimulation. In this case, both the low power and low noise characteristics of the recording function and the efficiency of the stimulation function may be secured without loss.

The signal path selector 115 may selectively connect one or more of the electrodes 121 to the first active chip 111 or the second active chip 112, based on whether the first function or the second function is performed. According to one or more embodiments, the signal path selector 115 may connect a first electrode of the electrodes 121 to the first active chip 111 to perform the first function and connect a second electrode of the electrodes 121, which is distinguished from the first electrode, to the second active chip 112 to perform the second function. For example, such a connection may be formed in an application in which different functions (e.g., the first function and the second function) are performed using different electrodes (e.g., the first electrode and the second electrode). According to one or more embodiments, the signal path selector 115 may connect the first electrode of the electrodes 121 to the first active chip 111 to perform the first function and connect the first electrode to the second active chip 112 to perform the second function. For example, such a connection may be formed in an application in which different functions (e.g., the first function and the second function) are performed using one electrode (e.g., the first electrode).

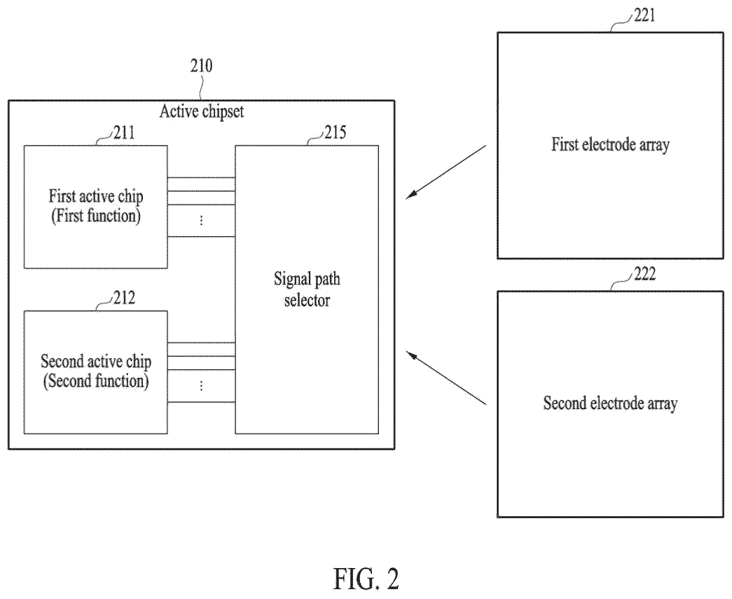

FIG. 2 is a diagram illustrating an example of the versatility of an active chipset, according to one or more embodiments. Referring to FIG. 2, an active chipset 210 may include a first active chip 211, a second active chip 212, and a signal path selector 215. The first active chip 211 may perform a first function in relation to a target, and the second active chip 212 may perform a second function in relation to the target.

The active chipset 210 may be universally used for a first electrode array 221 and a second electrode array 222. The first electrode array 221 may have properties dedicated to a first application. The second electrode array 222 may have properties dedicated to a second application that is distinguished from the first application.

According to one or more embodiments, the first function may be a measurement function, and the second function may be a stimulation function. The first application and the second application may perform the measurement function and the stimulation function with different electrode arrangements. The first electrode array 221 may perform the measurement function and the stimulation function through a first electrode arrangement, and the second electrode array 222 may perform the measurement function and the stimulation function through a second electrode arrangement that is distinguished from the first electrode arrangement.

For example, according to the first electrode arrangement, different electrodes may be used for the measurement function and the stimulation function, and according to the second electrode arrangement, the same electrode may be used for the measurement function and the stimulation function. In this case, when the first electrode array 221 is connected to the active chipset 210 to implement the first application, the signal path selector 215 may connect a first electrode of the first electrode array 221 to the first active chip 211 to perform the measurement function and connect a second electrode of the first electrode array 221, which is distinguished from the first electrode, to the second active chip 212 to perform the stimulation function. When the second electrode array 222 is connected to the active chipset 210 to implement the second application, the signal path selector 215 may connect a first electrode of the second electrode array 222 to the first active chip 211 to perform the measurement function and connect the first electrode of the second electrode array 222 to the second active chip 212 to perform the stimulation function. According to the first application of the first electrode arrangement, the measurement function and the stimulation function may be performed simultaneously, and according to the second application of the second electrode arrangement, the measurement function and the stimulation function may be performed at different times.

According to one or more embodiments, the first function may be a first measurement function using a first frequency range, and the second function may be a second measurement function using a second frequency range. The first application and the second application may perform measurement functions (e.g., the first measurement function and the second measurement function) using different frequency ranges (e.g., the first frequency range and the second frequency range).

For example, when the first electrode array 221 is connected to the active chipset 210 to implement the first application, the signal path selector 215 may connect electrodes of the first electrode array 221 to the first active chip 211 to perform the first measurement function. When the second electrode array 222 is connected to the active chipset 210 to implement the second application, the signal path selector 215 may connect electrodes of the second electrode array 222 to the second active chip 212 to perform the second measurement function.

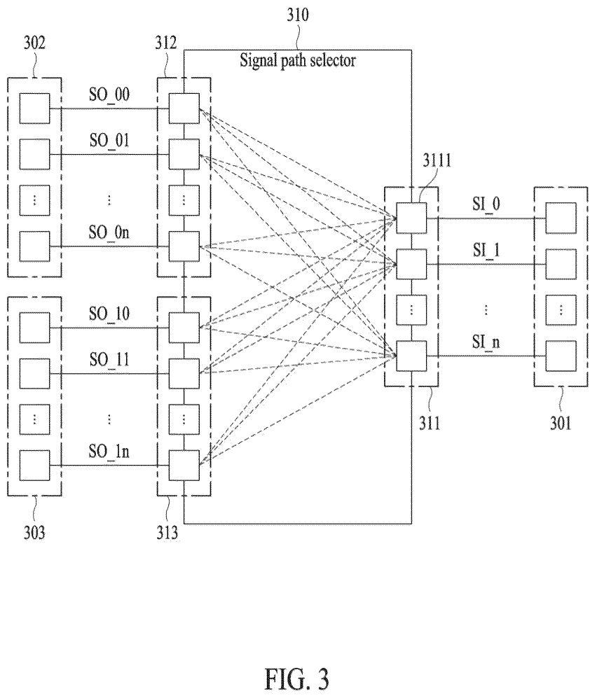

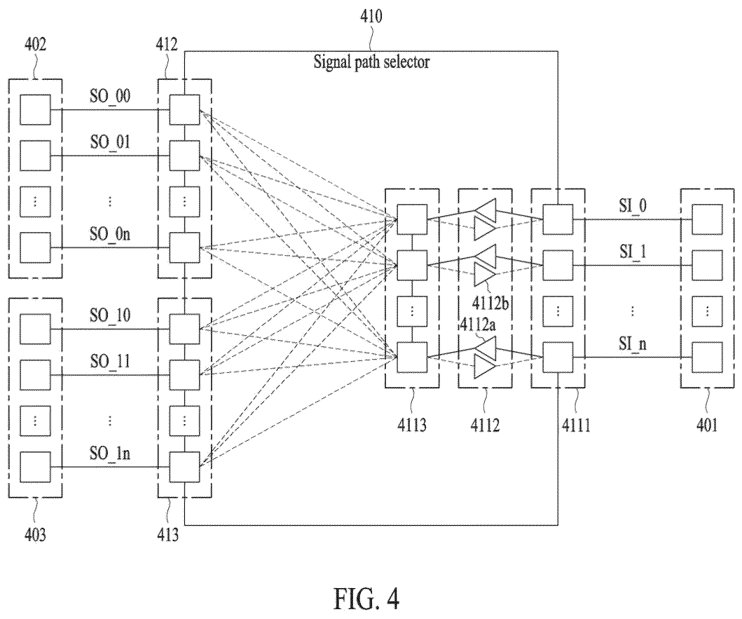

FIGS. 3 and 4 are diagrams illustrating examples of a configuration of a signal path selector, according to one or more embodiments. Referring to FIG. 3, a signal path selector 310 may include a path switch 311 selectively connecting one or more of electrodes 301 to a first active chip or a second active chip.

First signal terminals 302 of the first active chip may be connected to first chip-side terminals 312 of the signal path selector 310, and second signal terminals 303 of the second active chip may be connected to second chip-side terminals 313 of the signal selector 310. The signal path selector 310 may transmit input signals SI_0 to SI_n received from the electrodes 301 to the first active chip through the first chip-side terminals 312 and the first signal terminals 302 and to the second active chip through the second chip-side terminals 313 and the second signal terminals 303. The signal path selector 310 may transmit first output signals SO_00 to SO_0n received through the first signal terminals 302 and the first chip-side terminals 312 to the electrodes 301 and transmit second output signals SO_10 to SO_1n received through the second signal terminals 303 and the second chip-side terminals 313 to the electrodes 301.

According to one or more embodiments, the signal path selector 310 may form a base signal path fully connecting the first signal terminals 302 and the second signal terminals 303 to the path switch 311. The first signal terminals 302 and the second signal terminals 303 may be fully connected to individual switching elements (e.g., a first switching element 3111) of the path switch 311. For example, the individual switching elements 3111 may be multiplexers.

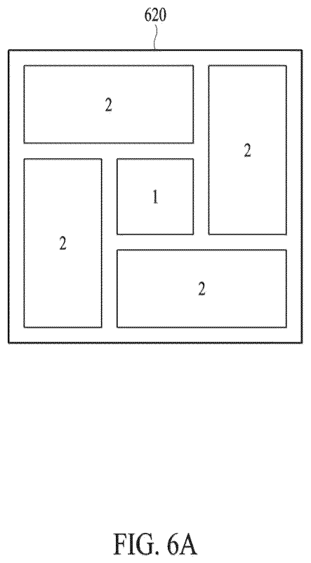

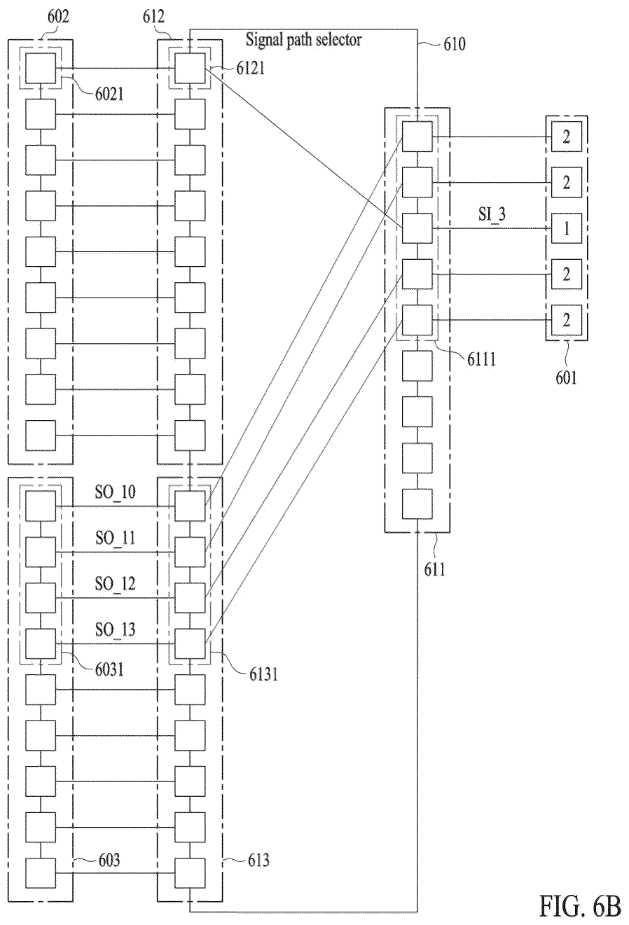

The base signal path may be at least partially used based on the characteristic of an electrode array. That is, the base signal path may include a plurality of signal paths. This may also be referred to as a plurality of partial signal paths. In other words, the base signal path may correspond to all available signal paths for connecting signal terminals to active chips, and the partial signal paths may refer to fewer than the total amount of all signal paths for connecting certain signal terminals to certain active chips. As an example, FIG. 3 shows the base signal path that include all available signal paths between the path switch 311, and the signal terminals 302/303, whereas FIG. 6B (described in further detail below) shows a partial signal path where five signal paths of all available signal paths are utilized.

For example, when the number of electrodes 301 is less than the number of individual switching elements, a partial path of the base signal path associated with partial individual switching elements that are not connected to the electrodes 301 may be deactivated. For example, when the first signal terminals 302 and the second signal terminals 303 are partially used, a partial amount of the base signal paths associated with unused terminals of the first signal terminals 302 and the second signal terminals 303 may be deactivated. In other words, the signal path selector 310 may include a base signal path as shown by the dotted lines in FIG. 3 between the path switch 311 and the terminals 302 and 303. When the number of electrodes 301 is less than the number of individual switching elements, fewer than the total number of signal paths of the base signal path may be used (i.e., a partial signal path). As multiple combinations of the total number of signal paths of the base signal path may be utilized, the base signal path may include a plurality of partial signal paths. As such, the signal paths that are associated with individual switching elements that are not connected to the electrodes being used for the corresponding function may be deactivated, and only a partial number of the total number of base signal paths may be utilized/activated (i.e., a partial signal path may be used). For example, a first number of signal paths of the base signal path may be used to be connected to a first electrode array, and a second number of signal paths of the base signal path may be used to be connected to a second electrode array. The signal paths of the first number of signal paths may be different from the signal paths of the second number of signal paths.

According to one or more embodiments, the signal path selector 310 may provide different signal paths for different applications. Each signal path may use all the base signal paths or may use a partial path of the base signal path. A signal path that uses a partial path of the base signal path may be referred to as a partial signal path. For example, a first electrode array for a first application and a second electrode array for a second application may each form the partial signal path, the partial signal path being a signal path that includes fewer than the total amount of signal paths of the base signal path. The signal path selector 310 may provide a first partial signal path of the base signal path to the first electrode array used for the first application and provide a second partial signal path of the base signal path, which is distinguished from the first partial signal path, to the second electrode array (e.g., another electrode array distinguished from the first electrode array) used for the second application.

Referring to FIG. 4, a signal path selector 410 may include path switches 4111 and 4113 and an amplifier 4112. The amplifier 4112 may include an input amplifier (e.g., an individual input amplifier 4112a) that amplifies input signals SI_0 to SI_n input from electrodes 401 and an output amplifier (e.g., an individual output amplifier 4112b) that amplifies first output signals SO_00 to SO_0n and second output signals SO_10 to SO_1n output from a first active chip and a second active chip. The first output signals SO_00 to SO_0n and the second output signals SO_10 to SO_1n may be received from the first active chip and the second active chip through first signal terminals 402 and second signal terminals 403 respectively, and first chip-side terminals 412 and second chip-side terminals 413 respectively.

The input signals SI_0 to SI_n, the first output signals SO_00 to SO_0n, and the second output signals SO_10 to SO_1n may be very small signals. The signal path selector 410 may increase the length of a signal path. The amplifier 4112 may compensate for signal attenuation that may occur due to the increase in the length of the signal path.

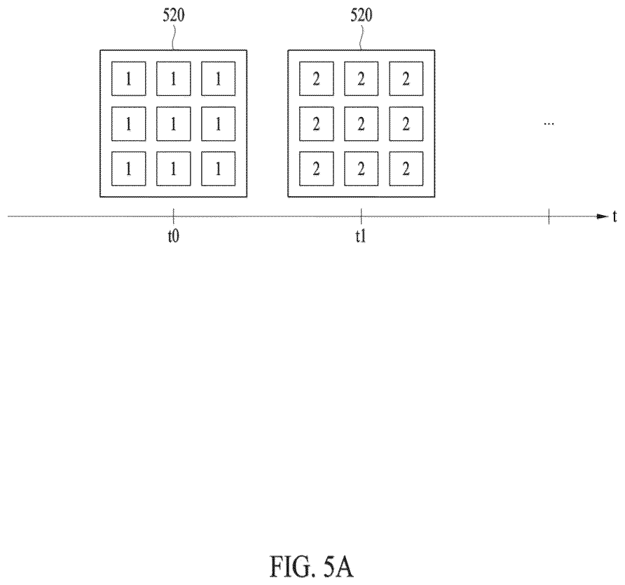

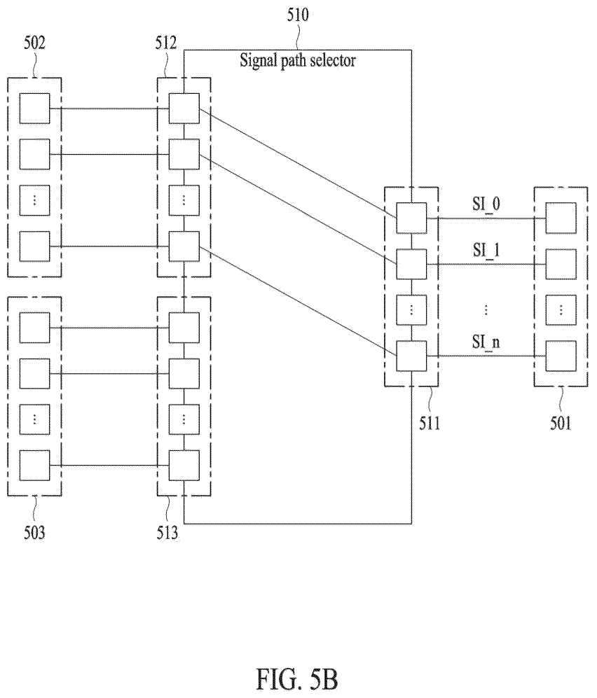

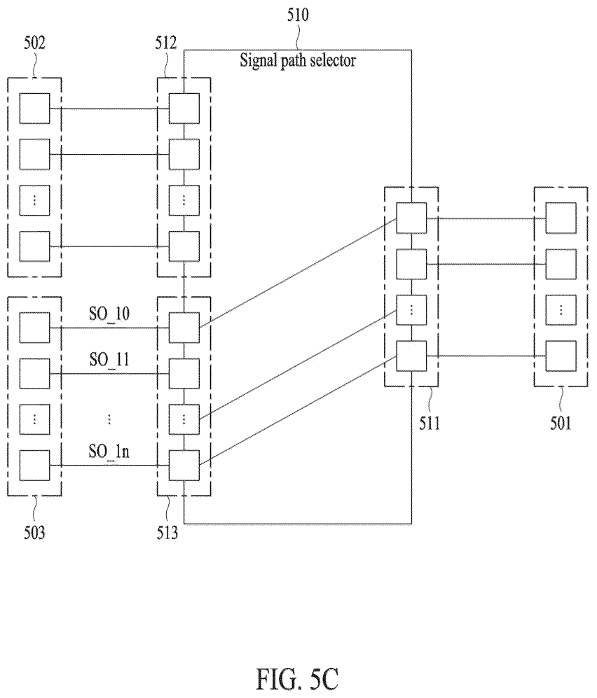

FIGS. 5A to 5C are diagrams illustrating examples of an application in which an electrode array is used for different functions, based on a time interval, according to one or more embodiments. Referring to FIG. 5A, an electrode array 520 may be used for a first function at a first time t0 and for a second function at a second time t1. The first function may be performed by a first active chip, and the second function may be performed by a second active chip.

Referring to FIGS. 5A and 5B, a signal path selector 510 may connect electrodes 501 to first signal terminals 502 of the first active chip to use the electrode array 520 for the first function at the first time t0. For example, a path switch 511 of the signal path selector 510 may connect the electrodes 501 to the first signal terminals 502 through first chip-side terminals 512. For example, the first function may be a measurement function, and input signals SI_0 to SI_n may be provided to the first active chip.

Referring to FIGS. 5A and 5C, the signal path selector 510 may connect the electrodes 501 to second signal terminals 503 of the second active chip to use the electrode array 520 for the second function at the second time t1. For example, the path switch 511 of the signal path selector 510 may connect the electrodes 501 to the second signal terminals 503 through second chip-side terminals 513. For example, the second function may be a stimulation function, and second output signals SO_10 to SO_1n may be provided to the electrodes 501. FIGS. 5B and 5C may show examples in which all base signal paths are used. In other words, all signal paths of the base signal path for connecting the electrodes 501 to the first signal terminals 502 may be used as shown in FIG. 5B, and all signal paths of the base signal path for connecting the electrodes 501 to the second signal terminals 503 may be used as shown in FIG. 5C. Although FIGS. 5B and 5C are described as all signal paths of the base signal path, this indicates all signal paths of the base signal path that correspond to the first signal terminals 502 and the second signal terminals 503, respectively. Thus, as shown in FIG. 5B, the partial signal path of the base signal path includes all signal paths of the base signal path that correspond to the first signal terminals 502, and as shown in FIG. 5C, the partial signal path of the base signal path includes all signal paths of the base signal path that correspond to the second signal terminals 503.

FIGS. 6A and 6B are diagrams illustrating examples of an application in which electrodes of an electrode array are used for different functions, based on a position in the electrode array, according to one or more embodiments. Referring to FIG. 6A, electrodes of an electrode array 620 may be used for a first function or a second function, based on a position in the electrode array 620. An electrode used for the first function may be referred to as a first electrode (labelled in FIGS. 6A and 6B as “1”), and an electrode used for the second function may be referred to as a second electrode (labelled in FIGS. 6A and 6B as “2”). For example, a first electrode 1 (or group of first electrodes) may be positioned in the center of the electrode array 620, and the second electrodes 2 (or groups of second electrodes) may be positioned around the first electrode 1. The first function may be performed by a first active chip, and the second function may be performed by a second active chip.

Referring to FIG. 6B, a signal path selector 610 may connect a first electrode 1 of electrodes 601 used for the first function to a first corresponding individual signal terminal 6021 of first signal terminals 602 of the first active chip. For example, a corresponding individual switching element of the path switch 611 of the signal path selector 610 may connect the first electrode 1 to the first corresponding individual signal terminal 6021 through a first corresponding individual chip-side terminal 6121 of first chip-side terminals 612. For example, the first function may be a measurement function, and an input signal SI_3 may be provided to the first active chip.

The signal path selector 610 may connect second electrodes 2 of the electrodes 601 used for the second function to second corresponding individual signal terminals 6031 of second signal terminals 603 of the second active chip. For example, corresponding individual switching elements of the path switches 611 of the signal path selector 610 may connect the second electrodes 2 to the second corresponding individual signal terminals 6031 through second corresponding individual chip-side terminals 6131 of second chip-side terminals 613. For example, the second function may be a stimulation function, and second output signals SO_10 to SO_13 may be provided to the second electrodes 2.

In one or more examples, as shown in FIG. 6B, a partial signal path of the base signal path may be used. In this case, individual switching elements other than corresponding individual switching elements 6111 in the path switch 611, individual chip-side terminals other than the first corresponding individual chip-side terminal 6121 of the first chip-side terminals 612, individual chip-side terminals other than the second corresponding individual chip-side terminals 6131 of the second chip-side terminals 613, individual signal terminals other than the first corresponding individual signal terminal 6021 of the first signal terminals 602, and individual signal terminals other than the second corresponding individual signal terminals 6031 of the second signal terminals 603 may not be used to form a connection path. In other words, as shown in FIG. 6B, a partial signal path of the base signal path (e.g., one signal path) may be used to connect electrode 1 of electrodes 601 to the first individual chip-side terminal 6121, and a partial signal path of base signal path (e.g., four signal paths) may be used to connect electrodes 2 of electrodes 601 to the second individual chip-side terminals 6131. The overall partial signal path of the base signal path in FIG. 6B may indicate all five paths shown. As shown in FIG. 6B, the number of signal paths connecting the electrode 1 is one, and the number of signal paths connecting electrodes 2 is four, and as such, it may be said that the partial signal path used for connection to the first individual chip-side terminal 6121 is different from the partial signal path used for connection to the second individual chip-side terminals 6131.

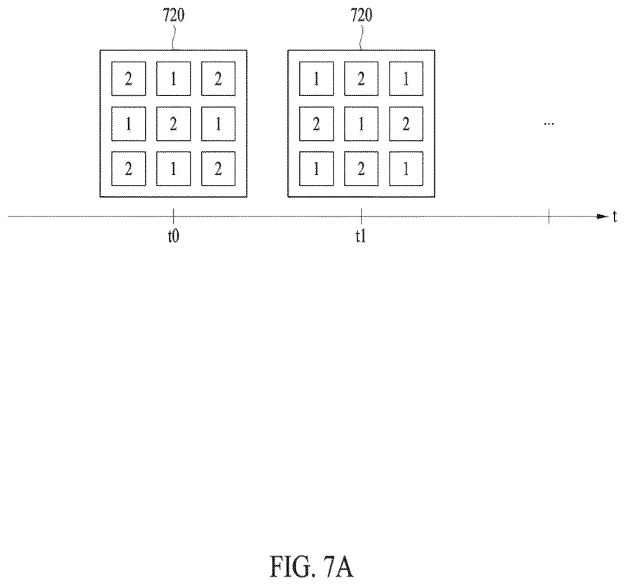

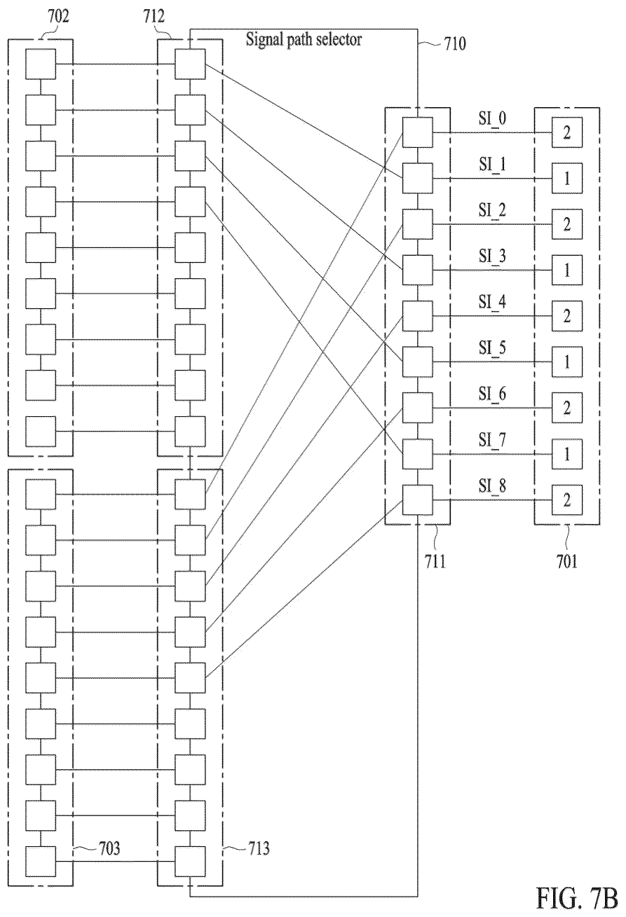

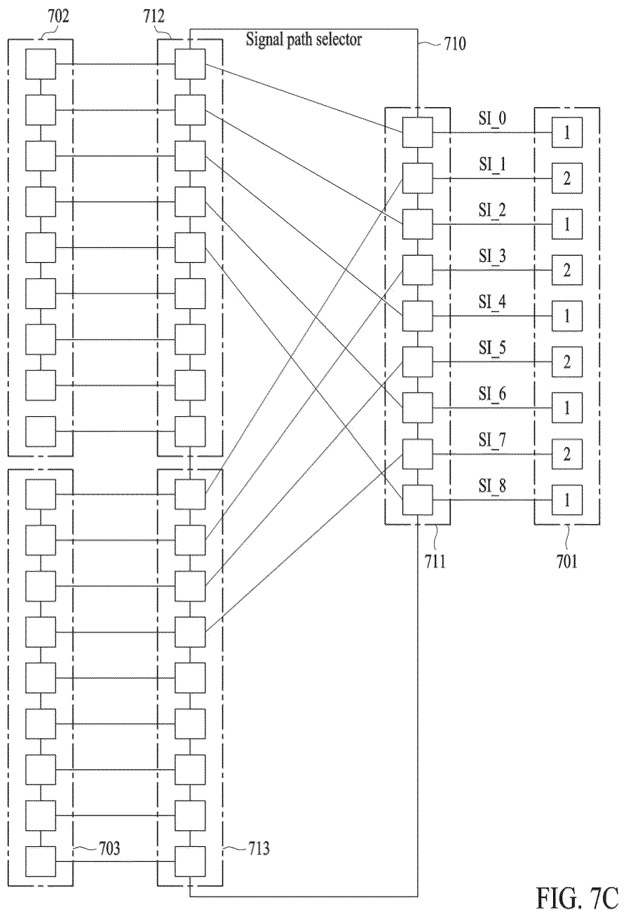

FIGS. 7A to 7C are diagrams illustrating examples of an application in which electrodes of an electrode array are used for different functions, based on a time interval, according to one or more embodiments. Referring to FIG. 7A, an electrode array 720 may be used for a first function and a second function with a first electrode arrangement at a first time t0 and used for the first function and the second function with a second electrode arrangement at a second time t1. The first function may be performed on a target by a first active chip, and the second function may be performed by a second active chip. The electrode arrangements may vary by position of electrodes in the electrode array 720. For example, in time to, the first electrodes 1 and the second electrodes 2 may have a first arrangement, and in time t1, the first electrodes 1 and the second electrodes 2 may have a second arrangement different from the first arrangement.

Referring to FIGS. 7A and 7B, a signal path selector 710 may connect electrodes 701 to first corresponding individual signal terminals of first signal terminals 702 of the first active chip and second corresponding individual signal terminals of second signal terminals 703 of the second active chip to use the electrode array 720 for the first function and the second function with the first electrode arrangement at the first time to. For example, a path switch 711 of the signal path selector 710 may connect the electrodes 701 to first corresponding individual signal terminals through first corresponding individual chip-side terminals of first chip-side terminals 712 and to second corresponding individual signal terminals of the second signal terminals 703 through second corresponding individual chip-side terminals of second chip-side terminals 713. For example, the first function may be a first measurement function using a first frequency range, and the second function may be a second measurement function using a second frequency range. Input signals SI_1, SI_3, SI_5, and SI_7 of the first frequency range may be provided to the first active chip, and input signals SI_0, SI_2, SI_4, SI_6, and SI_8 of the second frequency range may be provided to the second active chip.

Referring to FIGS. 7A and 7C, the signal path selector 710 may connect the electrodes 701 to the first corresponding individual signal terminals of the first signal terminals 702 of the first active chip and the second corresponding individual signal terminals of the second signal terminals 703 of the second active chip to use the electrode array 720 for the first function and the second function with the second electrode arrangement at the second time t1. Accordingly, input signals SI_0, SI_2, SI_4, SI_6, and SI_8 of the first frequency range may be provided to the first active chip, and input signals SI_1, SI_3, SI_5, and SI_7 of the second frequency range may be provided to the second active chip.

In one or more embodiments, as shown in FIGS. 7B and 7C, a partial signal path of the base signal path may be used. In this case (and similar to that described above), individual chip-side terminals other than the first corresponding individual chip-side terminals of the first chip-side terminals 712, individual chip-side terminals other than the second corresponding individual chip-side terminals of the second chip-side terminals 713, individual signal terminals other than the first corresponding individual signal terminals of the first signal terminals 702, and individual signal terminals other than the second corresponding individual signal terminals of the second signal terminals 703 may not be used to form a connection path. In other words, in FIG. 7B, the partial signal path may include four signal paths corresponding to four of the first chip-side terminals 712 and five signal paths corresponding to five of the second chip-side terminals 713. In FIG. 7C, the partial signal path may include five signal paths corresponding to five of the first chip-side terminals 712 and four signal paths corresponding to four of the second chip-side terminals 713. Alternatively, the structure of FIGS. 7B and 7C may be individually referred to as multiple partial signal paths. That is, FIG. 7B may include a first partial signal path that include four signal paths corresponding to four of the first chip-side terminals 712, and a second partial signal path that includes five signal paths corresponding to five of the second chip-side terminals 713. FIG. 7C may include a first partial signal path that includes five signal paths corresponding to five of the first chip-side terminals 712, and a second partial signal path that includes four signal paths corresponding to four of the second chip-side terminals 713.

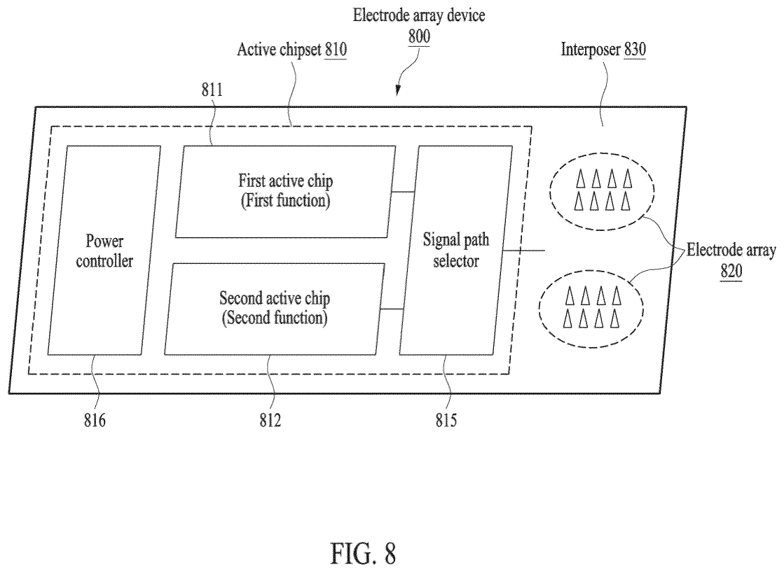

FIG. 8 is a diagram illustrating an example of packaging an electrode array device using an interposer, according to one or more embodiments. Referring to FIG. 8, an electrode array device 800 may include an active chipset 810, an electrode array 820, and an interposer 830. The active chipset 810 may include a first active chip 811, a second active chip 812, a signal path selector 815, and a power controller 816. The interposer 830 may connect (e.g., electrically connect) the active chipset 810 to the electrode array 820. The active chipset 810 and the electrode array 820 may be formed on the interposer 830.

According to one or more embodiments, electrodes of the electrode array 820 may be formed on the interposer 830 by processing the interposer 830. The electrodes may be made of the same material as the interposer 830 or different materials from the interposer 830. For example, the electrodes may be made of the same material as the interposer 830 and formed on the interposer 830 by processing the interposer 830 during the process of forming the interposer 830.

The power controller 816 may supply power to the first active chip 811 and the second active chip 812. The power controller 816 may supply the power to the first active chip 811 and/or the second active chip 812 at the timing when an operation of the first active chip 811 and/or the second active chip 812 is required. For example, the first active chip 811 may be activated to perform a first function, and the second active chip 812 may be activated to perform a second function. At the timing when the first function is performed and the second function is not performed, the power controller 816 may supply the power to the first active chip 811 and cut off the power supply to the second active chip 812. At the timing when the second function is performed and the first function is not performed, the power controller 816 may supply the power to the second active chip 812 and cut off the power supply to the first active chip 811. Due to the power controller 816, power consumption may be reduced, and heat generation due to the operation of the first active chip 811 and the second active chip 812 may be reduced.

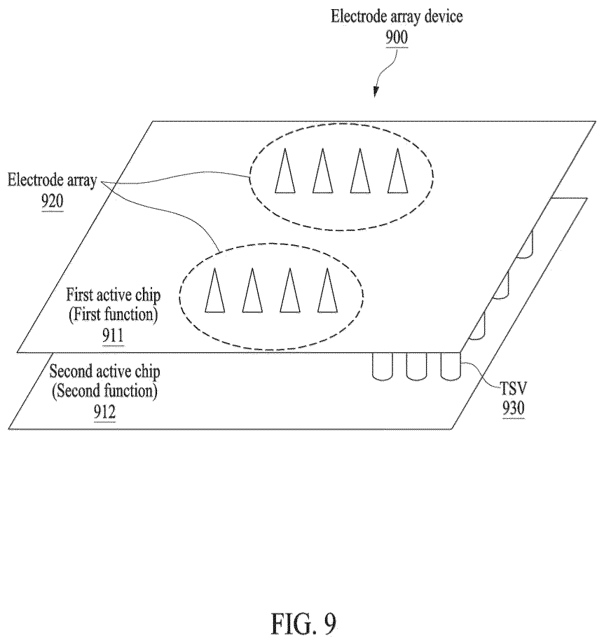

FIG. 9 is a diagram illustrating an example of packaging an electrode array device using vertical stacking, according to one or more embodiments. Referring to FIG. 9, an electrode array device 900 may include a first active chip 911, a second active chip 912, and an electrode array 920. The first active chip 911 may be vertically stacked on the second active chip 912. The electrode array 920 may be formed on the first active chip 911.

For ease of description, a signal path selector is omitted from FIG. 9, but the electrode array device 900 may include the signal path selector. The signal path selector may be electrically connected to the first active chip 911 and the second active chip 912 through a through silicon via (TSV) 930. The TSV 930 may form portions or all of a signal path formed by the signal path selector. Due to the TSV 930, the length of the signal path connecting the first active chip 911, the second active chip 912, the signal path selector, and the electrode array 920 may be shortened, and a signal attenuation phenomenon may be alleviated. According to one or more embodiments, when the TSV 930 is applied to the electrode array device 900, an amplifier may be omitted from the signal path selector.

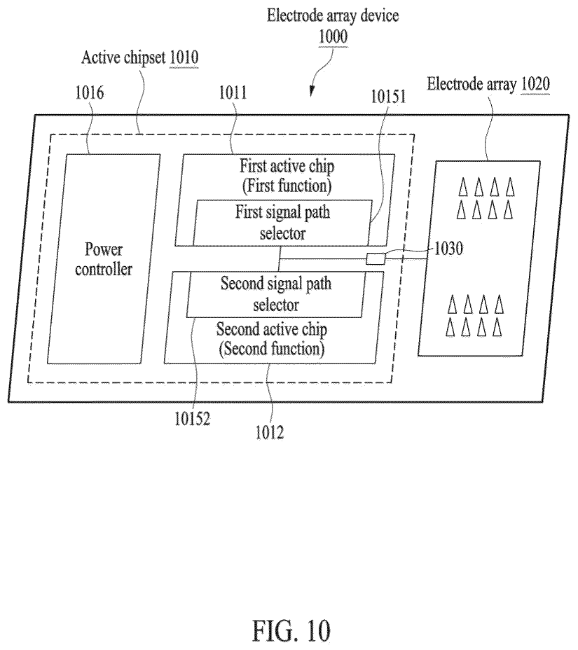

FIG. 10 is a diagram illustrating an example of the arrangement of signal path selectors of an electrode array device, according to one or more embodiments. Referring to FIG. 10, an electrode array device 1000 may include an active chipset 1010 and an electrode array 1020. The active chipset 1010 may include a first active chip 1011, a second active chip 1012, a first signal path selector 10151, a second signal path selector 10152, and a power controller 1016.

The first signal path selector 10151 may be implemented in the first active chip 1011, and the second signal path selector 10152 may be implemented in the second active chip 1012. The first signal path selector 10151 may form a first signal path corresponding to performance of a first function of the first active chip 1011, and the second signal path selector 10152 may form a second signal path corresponding to performance of a second function of the second active chip 1012. According to one or more embodiments, a path switch 1030 may be positioned between the first signal path selector 10151, the second signal path selector 10152, and the electrode array 1020. The path switch 1030 may connect the first signal path selector 10151 to the electrode array 1020 when the first function is performed and connect the second signal path selector 10152 to the electrode array 1020 when the second function is performed.

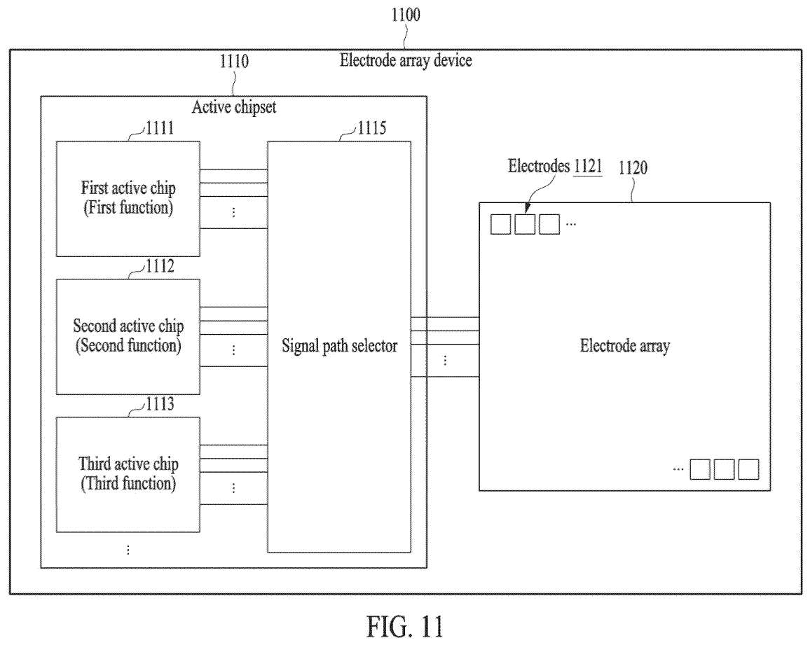

FIG. 11 is a diagram illustrating an example of three or more active chips being used in an electrode array device, according to one or more embodiments. Referring to FIG. 11, an electrode array device 1100 may include an electrode array 1120 including electrodes 1121 contacting a target, a first active chip 1111 configured to perform a first function in relation to the target using the electrode array 1120, a second active chip 1112, which is distinguished from the first active chip 1111, configured to perform a second function in relation to the target using the electrode array 1120, which is distinguished from the first function, a third active chip 1113 that is distinguished from the first active chip 1111 and the second active chip 1112 and configured to perform a third function in relation to the target, which is distinguished from the first function and the second function, using the electrode array 1120, and a signal path selector 1115 configured to selectively connect one or more of the electrodes 1121 to the first active chip 1111, the second active chip 1112, or the third active chip 1113, based on whether the first function, the second function, or the third function is performed.

FIG. 11 shows an example in which an active chipset 1110 includes three active chips (e.g., the first active chip 1111, the second active chip 1112, and the third active chip 1113) but embodiments are not limited thereto. For example, the active chipset 110 may include two active chips.

A signal path selector 1115 may connect a first electrode of the electrodes 1121 to the first active chip 1111 to perform the first function and may connect a second electrode of the electrodes 1121, which is distinguished from the first electrode, to the second active chip 1112 to perform the second function.

The signal path selector 1115 may connect the first electrode of the electrodes 1121 to the first active chip 1111 to perform the first function and connect the first electrode to the second active chip 1112 to perform the second function.

The signal path selector 1115 may connect any of the first electrode of the electrodes 1121, the second electrode of the electrodes 1121, and/or a third electrode(s) of the electrodes 1121 to the third active chip 1113 to perform the third function.

The signal path selector 1115 may include a path switch selectively connecting one or more of the electrodes 1121 to the first active chip 1111, the second active chip 1112, or the third active chip 1113.

The signal path selector 1115 may further include an input amplifier configured to amplify an input signal input from one or more of the electrodes 1121 and an output amplifier configured to amplify an output signal output from the first active chip 1111 or the second active chip 1112.

The signal path selector 1115 may form a base signal path fully connecting first signal terminals of the first active chip 1111, second signal terminals of the second active chip 1112, and third signal terminals of the third active chip 1113 to the path switch. The base signal path may be partially used based on the characteristic of the electrode array 1120. That is, the base signal path may include a plurality of partial signal paths that may be used based on the characteristic of the electrode array 1120.

The signal path selector 1115 may provide a first partial signal path of the base signal path (i.e., a first number of signal paths among the signal paths of the overall base signal path) to the electrode array 1120, and provide a second partial signal path (i.e., a second number of signal paths among the signal paths of the overall base signal path) of the base signal path, which is distinguished from the first partial signal path, to another electrode array, which is distinguished from the electrode array 1120.

The first function and the second function may correspond to a measurement function and a stimulation function, respectively. The first function and the second function may correspond to the measurement function, and a third function may correspond to the stimulation function.

The first function may correspond to a first measurement function using a first frequency range, and the second function may correspond to a second measurement function using a second frequency range that is distinguished from the first frequency range. The first function may correspond to the first measurement function using the first frequency range, the second function may correspond to the second measurement function using the second frequency range that is distinguished from the first frequency range, and the third function may correspond to the stimulation function.

The first active chip 1111, the second active chip 1112, and the third active chip 1113 may be produced from separate semiconductor dies.

The first active chip 1111, the second active chip 1112, the third active chip 1113, and the signal path selector 1115 may form the active chipset 1110, and the active chipset 1110 and the electrode array 1120 may be formed on an interposer connecting the active chipset 1110 to the electrode array 1120.

The electrodes 1121 may be formed on the interposer by processing the interposer.

The first active chip 1111 may be vertically stacked on the second active chip 1112, the second active chip 1112 may be vertically stacked on the third active chip 1113, and the signal path selector 1115 may be electrically connected to the first active chip 1111, the second active chip 1112 and the third active chip 1113 through a TSV.

The electrode array 1120 may be formed on the first active chip 1111.

Various embodiments as set forth herein may be implemented as software including one or more instructions that are stored in a storage medium that is readable by a machine. For example, a processor of the machine may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

A processing device may be implemented using one or more general-purpose or special-purpose computers, such as, for example, a processor, a controller, an arithmetic logic unit (ALU), a digital signal processor (DSP), a microcomputer, a field programmable gate array (FPGA), a programmable logic unit (PLU), a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciate that a processing device may include multiple processing elements and/or multiple types of processing elements. For example, the processing device may include a plurality of processors, or a single processor and a single controller. In addition, different processing configurations are possible, such as parallel processors.

The software may include a computer program, a piece of code, an instruction, or some combination thereof, to independently or uniformly instruct or configure the processing device to operate as desired. Software and data may be stored in any type of machine, component, physical or virtual equipment, or computer storage medium or device capable of providing instructions or data to or being interpreted by the processing device. The software also may be distributed over network-coupled computer systems so that the software is stored and executed in a distributed fashion. The software and data may be stored by one or more non-transitory computer-readable recording mediums.

The methods according to the above-described embodiments may be recorded as program instructions in non-transitory computer-readable media to implement various operations of the above-described embodiments. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of non-transitory computer-readable media include magnetic media such as hard disks, floppy disks, and magnetic tape; optical media such as CD-ROM discs and/or DVDs; magneto-optical media such as optical discs; and hardware devices that are specially configured to store and perform program instructions, such as read-only memory (ROM), random access memory (RAM), flash memory, and the like. Examples of program instructions include both machine code, such as produced by a compiler, and files containing higher-level code that may be executed by the computer using an interpreter.

The above-described hardware devices may be configured to act as one or more software modules in order to perform the operations of the above-described embodiments, or vice versa.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. An electrode array device comprising:
a first electrode array comprising a plurality of electrodes configured to contact a target;
a first active chip configured to perform, with the first electrode array, a first measurement function on the target using a first frequency range and receive a first input signal corresponding to the first frequency range from the first electrode array;
a second active chip, which is structurally different from the first active chip, configured to perform, with the first electrode array, a second measurement function on the target using a second frequency range that is different from the first frequency range and receive a second input signal corresponding to the second frequency range from the first electrode array; and
a signal path selector configured to selectively connect at least one electrode of the plurality of electrodes to the first active chip in the case that the first active chip is to perform the first measurement function or to the second active chip in the case that the second active chip is to perform the second measurement function.

2. The electrode array device of claim 1, wherein the signal path selector is further configured to:
connect a first electrode of the plurality of electrodes to the first active chip in the case that the first measurement function is to be performed; and
connect a second electrode of the plurality of electrodes that is different from the first electrode to the second active chip in the case that the second measurement function is to be performed.

3. The electrode array device of claim 1, wherein the signal path selector is further configured to:
connect a first electrode of the plurality of electrodes to the first active chip in the case that the first measurement function is to be performed; and
connect the first electrode of the plurality of electrodes to the second active chip in the case that the second measurement function is to be performed.

4. The electrode array device of claim 1, wherein the signal path selector comprises a path switch configured to selectively connect the at least one electrode to the first active chip in the case that the first measurement function is to be performed and the second active chip in the case that the second measurement function is to be performed.

5. The electrode array device of claim 4, wherein the signal path selector further comprises:
an input amplifier configured to amplify an input signal input from the at least one electrode; and
an output amplifier configured to amplify an output signal output from the first active chip or the second active chip.

6. The electrode array device of claim 5, wherein the signal path selector comprises a base signal path configured to fully connect, to the path switch, first signal terminals of the first active chip and second signal terminals of the second active chip, and
wherein the base signal path comprises a plurality of partial signal paths.

7. The electrode array device of claim 6, wherein the signal path selector is further configured to:
connect a first partial signal path of the plurality of partial signal paths to the first electrode array; and
connect a second partial signal path of the plurality of partial signal paths to a second electrode array that is different from the first electrode array, and
wherein the first partial signal path is different from the second partial signal path.

8. The electrode array device of claim 1, wherein the first frequency range is in a range of 100 Hz to 20 kHz, and
wherein the second frequency range is in a range of direct current (DC) to 100 Hz.

9. The electrode array device of claim 1, wherein the first input signal comprises an action potential (AP) signal, and
wherein the second input signal comprises at least one of a local-field potential (LFP) signal, a potential of hydrogen (pH) and/or electrical conversion signal, an ion and/or electrical conversion signal, and a combination thereof.

10. The electrode array device of claim 1, wherein the first active chip and the second active chip are produced from separate semiconductor dies.

11. The electrode array device of claim 1, wherein the first active chip, the second active chip, and the signal path selector are configured as an active chipset, and
wherein the electrode array device further comprises an interposer connecting the active chipset to the first electrode array.

12. The electrode array device of claim 11, wherein the interposer comprises the plurality of electrodes.

13. The electrode array device of claim 1, wherein the first active chip is vertically stacked on the second active chip, and
wherein the signal path selector is connected to the first active chip and the second active chip through a through silicon via (TSV).

14. The electrode array device of claim 13, wherein the first electrode array is on the first active chip.

15. An electrode array device comprising:
an electrode array comprising a plurality of electrodes configured to contact a target;
a first active chip configured to perform, with the electrode array, a first measurement function on the target using a first frequency range;
a second active chip configured to perform, with the electrode array, a second measurement function on the target using a second frequency range that is different from the first frequency range;
a third active chip configured to perform, with the electrode array, a stimulation function on the target; and
a signal path selector configured to selectively connect at least one electrode of the plurality of electrodes to the first active chip in the case that the first active chip is to perform the first measurement function, the second active chip in the case that the second active chip is to perform the second measurement function, and the third active chip in the case that the third active chip is to perform the stimulation function.

16. The electrode array device of claim 15, wherein the signal path selector is further configured to:
connect a first electrode of the plurality of electrodes to the first active chip in the case that the first measurement function is to be performed; and
connect a second electrode of the plurality of electrodes that is different from the first electrode to the second active chip in the case that the second measurement function is to be performed.

17. The electrode array device of claim 15, wherein the signal path selector is further configured to:
connect a first electrode of the plurality of electrodes to the first active chip in the case that the first measurement function is to be performed; and
connect the first electrode to the second active chip in the case that the second measurement function is to be performed.

18. The electrode array device of claim 15, wherein the signal path selector comprises a path switch configured to selectively connect the at least one electrode to the first active chip in the case that the first measurement function is to be performed, the second active chip in the case that the second measurement function is to be performed, and the third active chip in the case that the stimulation function is to be performed.

19. The electrode array device of claim 18, wherein the signal path selector comprises a base signal path configured to fully connect, to the path switch, first signal terminals of the first active chip, second signal terminals of the second active chip, and third signal terminals of the third active chip, and
wherein the base signal path comprises a plurality of partial signal paths.

20. An electrode array device comprising:
an electrode array comprising a plurality of electrodes configured to contact a target;
a first active chip configured to perform, with the electrode array, a first function on the target;
a second active chip that is structurally different from the first active chip and configured to perform, with the electrode array, a second function on the target, the second function being different from the first function;
a third active chip that is structurally different from the first active chip and the second active chip and configured to perform, with the electrode array, a third function on the target, the third function being different from the first function and the second function; and
a signal path selector configured to selectively connect at least one electrode of the plurality of electrodes to the first active chip in the case that the first function is to be performed, the second active chip in the case that the second function is to be performed, and the third active chip in the case that the third function is to be performed.
